(19) United States
(12) Patent Application Publication
(10) Pub. No.: US 2014/0159921 A1
(43) Pub. Date: Jun. 12, 2014

(54) Configurable, Portable Patient Monitoring System

(71) Applicant: Spacelabs Healthcare LLC, Snoqualmie, WA (US)

(72) Inventors: Bruce Qualey, Mill Creek, WA (US); Scott Britt, Issaquah, WA (US); Nityanand Shetty, Secunderabad (IN)

(21) Appl. No.: 13/895,270

(22) Filed: May 15, 2013

Related U.S. Application Data
(63) Continuation-in-part of application No. 13/300,462, filed on Nov. 18, 2011.
(60) Provisional application No. 61/647,361, filed on May 15, 2012.
(60) Provisional application No. 61/415,799, filed on Nov. 19, 2010.

(51) Int. Cl. A61B 5/00; H05K 7/00

(52) U.S. Cl. CPC A61B 5/0015; H05K 7/00
USPC 340/870.09; 361/679.01

(57) ABSTRACT

A system for patient monitoring includes a plurality of components including a monitor and display assembly, optional stand-alone displays, optional stand-alone monitors, one or more modules, and at least one patient parameter measuring device. The display includes a flat glass front with a blackened border that appears continuous but allows the passage of light during alarm situations. The display functions as a touchscreen and includes a portion for alarm volume control. The system also includes a docking station for the monitor and display assembly and capnography and/or multigas pods for attachment to the monitor and display assembly. The monitor and display assembly, docking station, and pods enhance portability of the system. The monitor and display assembly, module(s), and patient parameter measuring device(s) are all interconnected via Dual Serial Bus (DSB) interfaces.

### CROSS-REFERENCE TO RELATED APPLICATIONS

The present specification relies on U.S. Provisional Patent Application No. 61/647,361, filed on May 15, 2012, for priority.

The present specification is also a continuation-in-part of U.S. patent application Ser. No. 13/300,462, of the same title, and filed on Nov. 18, 2011 which claims priority from U.S. Provisional Patent Application No. 61/415,799, entitled “Patient Monitoring System with Dual Serial Bus (DSB) Interface” and filed on Nov. 19, 2010, which are both herein incorporated by reference in their entirety.

Co-pending U.S. patent application Ser. No. 13/300,478, entitled “Dual Serial Bus Interface”, filed on Nov. 18, 2011 and assigned to the applicant of the present invention, is also herein incorporated by reference in its entirety.

### FIELD

The present specification relates generally to hospital-based patient monitoring systems. More particularly, the present specification relates to a configurable patient monitoring system comprised of a monitor and display assembly, optional stand-alone displays, optional stand-alone monitors, one or more modules, and a plurality of devices to measure patient parameters.

### BACKGROUND

A patient monitoring system is an electronic medical device that measures a patient's various vital signs, collects and processes all measurements as data, and then displays the data graphically and/or numerically on a viewing screen. Graphical data is displayed continuously as data channels on a time axis (waveforms). Patient monitoring systems are positioned near hospital beds, typically in critical care units, where they continually monitor patient status via measuring devices attached to the patient and can be viewed by hospital personnel. Some patient monitoring systems can only be viewed on a local display, whereas others can be joined to a network and thereby display data at other locations, such as central monitoring or nurses' stations.

Portable patient monitoring systems are available for use by emergency medical services (EMS) personnel. These systems typically include a defibrillator along with the monitor. Other portable units, such as Holter monitors, are worn by patients for a particular time period and then returned to the physician for evaluation of the measured and collected data. Current patient monitoring systems are able to measure and display a variety of vital signs, including, pulse oximetry (SpO₂), electrocardiograph (ECG), invasive blood pressure (IBP), non-invasive blood pressure (NIBP), electroencephalograph (EEG), body temperature, cardiac output, capnography (CO₂), mixed venous oxygen saturation (SvO₂), bispectral index (BISx), and respiration. Patient monitoring systems are capable of measuring and displaying maximum, minimum, and average values and frequencies, such as pulse and respiratory rates.

Data collected can be transmitted through fixed wire connections or wireless data communication. Power to patient monitoring systems can be supplied through a main power line or by batteries. While current patient monitoring systems are effective in monitoring patient conditions and notifying medical personnel of changes, they are not without certain drawbacks and limitations.

Patient monitoring systems are typically equipped with audio and visual alarms to notify medical personnel of changes in the patient's status. The alarm parameters can be set by the medical personnel. Audible nurse alarms can often be too loud and distracting to other patients and personnel. Bright, flashing visual nurse alarms can also be distracting to other patients. Conversely, more subtle visual nurse alarms can be too difficult to visualize, which can be a result of visual clutter on the monitoring system display or because the visual alarm is not differentiated enough from other information on the display. In addition, it can be difficult for nurses to silence an active alarm, delaying care to the patient. The typical user interface for alarm control is operated via traditional push-buttons or in many instances a touchscreen or keyboard.

Therefore, a need exists for a better alarm mechanism within patient monitoring systems, in which both the audible and visual alarms are easily recognized by the nurses while not disturbing patients. In addition, there is a need for an alarm mechanism in which an attending nurse can quickly silence the alarm and then focus on the patient's needs.

Current patient monitoring systems are traditionally bundled into an integrated package that includes the display, enclosure, and electronics. This limits flexibility and prevents users from customizing the monitoring system to their specific needs and available space. Therefore, a need exists for a modular patient monitoring system in which the individual components are discrete and can be connected in various configurations. Specifically, a need exists for a monitor that does not have an integrated display and can connect to a custom or commercial, off-the-shelf (COTS) display. Such a monitoring system would enable users to position the display and monitor in the most efficient manner, thereby freeing up valuable area in the patient vicinity.

### SUMMARY

The present specification is directed toward a configurable patient monitoring system comprised of a plurality of non-integrated components including a display, a monitor, one or more modules, and at least one patient parameter measuring device. A variety of patient parameters can be monitored and the parameter measuring devices are connected to the system via Dual Serial Bus (DSB) connectors and DSB cables.

In one embodiment, the present specification is directed toward a display device for use in patient monitoring systems, comprising: a housing having a front face and defining an enclosure, wherein said enclosure comprises a first opening on a right side of said housing and a second opening on a left side of said housing; a touchscreen mounted to the front of said housing, wherein said touchscreen comprises a flat piece of glass having a central display area and a black border that extends along a left, right, top, and bottom edge of said glass; a processor for determining an alarm state; and, light sources within said touchscreen which are activated by said processor during the alarm state, wherein said light sources are configured to pass through said black border and concurrently pass through said first opening and second opening.

In one embodiment, the display device further comprises a single prominent, programmable capacitive button along the border of said touchscreen. In one embodiment, the button comprises a metal capacitive piece. In another embodiment, the display device includes a section of the touchscreen programmed for control of the alarm light.

In one embodiment, the alarm lights are configurable by a user to define the minimum level of alarm lights that may be activated independent of the on-screen alarm display and/or alarm audio.

In one embodiment, the black border of the display device is silk-screened on the back of the glass. In another embodiment, the black border of the display device is comprised of an ink that is silk-screened or sprayed onto a masked out border area on the back of the glass. In another embodiment, the black border of the display device contains small apertures that make the border appear continuous and uniform but allow light to pass through.

In one embodiment, the light sources which emit light passing through the black border are the same light sources which emit light passing through the first opening and second opening.

In one embodiment, the light sources which emit light passing through the black border are different than the light sources which emit light passing through the first opening and second opening.

In another embodiment, the alarm lights are configured as a single nurse light across the top of the display. Additionally or optionally, in an embodiment, another nurse alarm light is positioned at the rear to provide complete nurse light visibility from any angle or position.

In another embodiment, the present specification is directed towards a system for patient monitoring comprising: at least one patient monitor that allows for communication with external devices, wherein said patient monitor is in electronic communication with and drives at least one display, and wherein said display comprises: a housing having a front and defining an enclosure, wherein said enclosure comprises a first opening on a right side of said housing and a second opening on a left side of said housing; a touchscreen mounted to the front of said housing, wherein said touchscreen comprises a flat piece of glass having a central display area and a black border that extends along a left, right, top, and bottom edge of said glass; at least one module for providing measurements of a plurality of patient parameters, wherein said module is in electronic communication with said patient monitor and wherein said module comprises at least one interface for electronically communicating with at least one patient parameter measurement device; a processor for determining an alarm state; and, light sources within said touchscreen which are activated by said processor during the alarm state, wherein said light sources are configured to pass through said black border and concurrently pass through said first opening and second opening; and at least one Dual Serial Bus (DSB) interface for enabling electronic communication between the patient monitor, module, and/or patient parameter measuring device.

The present specification is also directed toward a system for patient monitoring comprising: at least one patient monitor that allows for communication with external devices, wherein said patient monitor is in electronic communication with and drives at least one display, and wherein said display comprises: a housing having a front and a back; a touchscreen mounted to the front of said housing, wherein said touchscreen comprises a flat piece of glass having a central display area and a black border that extends along a left, right, top, and bottom edge of said glass, further wherein said monitor is fixedly attached to said back of said display; at least one module for providing measurements of a plurality of patient parameters, wherein said module is in electronic communication with said patient monitor and wherein said module comprises at least one interface for electronically communicating with at least one patient parameter measurement device; a processor for determining an alarm state; and, light sources within said touchscreen which are activated by said processor during the alarm state, wherein said light sources are configured to pass through said black border; and at least one Dual Serial Bus (DSB) interface for enabling electronic communication between the patient monitor, module, and/or patient parameter measuring device.

In one embodiment, the at least one light source is positioned proximate said top edge of said front face of said display device. In one embodiment, the touchscreen comprises an area corresponding to said light source for controlling alarm volume level. In one embodiment, said area comprises a first portion for decreasing said alarm volume level and a second portion for increasing said alarm volume level.

In one embodiment, at least one light source is positioned on said rear face of said display.

In one embodiment, said at least one patient monitor comprises a removable internal chassis for mounting a plurality of circuit boards.

In one embodiment, said at least one patient monitor comprises a handle attached to said patient monitor and wherein said handle further comprises an up and a down position, a set-point for balancing said patient monitor perpendicular to the floor when said patient monitor is carried using said handle and, a damper to retard downward motion of said handle when said handle is released from the up position.

In one embodiment, said at least one patient monitor comprises Li-Ion batteries and a microcontroller for monitoring charging, discharging and over-temperature conditions of said batteries. In one embodiment, said at least one patient monitor is capable of operating 8 hours on battery power while monitoring ECG, NIBP every 15 minutes and taking a recording every 15 minutes.

In one embodiment, said at least one patient monitor has a housing of Sabic Lexan EXL plastic. In one embodiment, said at least one patient monitor weighs less than 9 pounds.

The present specification is also directed toward a docking station having a receiving surface to receive a monitor and display device of a patient monitoring system, said monitor and display device having a first connector and a first plurality of monitor receptacles for transmission of digital information and power, said docking station comprising: a second plurality of receptacles; a second connector positioned on said receiving surface of said docking station for mating with said first connector of said monitor and display device; a circuit board for controlling said transmission of digital information and power; a molded recess matching an external shape of a bottom portion of said monitor and display device; at least one latching mechanism for securely holding said monitor and display device in place and, a release button for disengaging said latching mechanism for removal of said monitor and display device from said docking station; wherein, when said monitor and display device is securely mounted on said docking station via said latching mechanism, said first connector is in electrical communication with said second connector, further wherein said circuit board transfers said transmission of digital information and power away from said first plurality of receptacles, through said first and second connectors, and to said second plurality of receptacles.

In one embodiment, said second plurality of receptacles comprises Ethernet connection, DVI for external display, USB, serial ports, external nurse alert/external audio/IR receiver, power and SDLC (synchronous data link control) port.

In one embodiment, said docking station covers said first plurality of receptacles on said monitor and display device when the said monitor and display device is docked in the docking station.

In one embodiment, said monitor and display device further comprises a first plurality of vents and said docking station further comprises a second plurality of vents, said first and second plurality of vents aligning when said monitor and display device is mounted in said docking station.

In one embodiment, said molded recess further comprises an outward bevel to guide the monitor and display device into position during docking and at least one pin configured to snugly fit at least one corresponding opening on said monitor and display device to further guide placement of said monitor and display device into said docking station.

In one embodiment, the docking station further comprises at least one pin configured to fit at least one corresponding opening on said monitor and display device to further guide placement of said monitor and display device into said docking station.

In one embodiment, said release button is backlit when the monitor and display device is docked in the docking station.

The present specification is also directed toward an externally mountable pod for attaching to a monitor of a patient monitoring system, said pod comprising: a plurality of pogo pins for mating with connectors on said monitor; at least one guide pin for mating said pod with said monitor; a latching mechanism for connecting and removing said pod to and from said monitor; a button for actuating said latching mechanism; and, a plurality of receptacles on a side of said pod.

In one embodiment, said pod is a sidestream capnography or multigas pod.

In one embodiment, the pogo pins enable the pod to receive power from said monitor and enable communication between said pod and said monitor.

In one embodiment, said receptacles comprise inlet and scavenging ports.

The aforementioned and other embodiments of the present invention shall be described in greater depth in the drawings and detailed description provided below.

### BRIEF DESCRIPTION OF THE DRAWINGS

These and other objects and advantages of the present specification will become more fully apparent from the following detailed description when read in conjunction with the accompanying drawings with like reference numerals indicating corresponding parts throughout, wherein:

FIG. 1 is a block diagram depicting one embodiment of an exemplary configuration of the components of the patient monitoring system of the present specification, illustrating the use of Dual Serial Bus (DSB) cables to connect patient parameter measuring devices to the monitor;

FIG. 2A is an oblique side view illustration of one embodiment of a monitor and display assembly of the patient monitoring system;

FIG. 2B is an oblique side view illustration of one embodiment of the monitor and a portion of the display of a monitor and display assembly, depicting a rechargeable battery partially removed from the monitor;

FIG. 2C is a side view illustration of one embodiment of a monitor and display assembly illustrating a handle in an up position;

FIG. 2D is a rear view illustration of one embodiment of a monitor and display assembly depicting a plurality of receptacles;

FIG. 3 is a front view illustration of one embodiment of the monitor and display assembly of the patient monitoring system depicting a red alarm light on the front of the display;

FIG. 4 is an oblique inside-view illustration of an embodiment of a monitor and display assembly with circuit boards mounted onto a removable internal chassis;

FIG. 5 is an oblique front view illustration of one embodiment of a quick release mount;

FIG. 6 is an oblique front view illustration of one embodiment of an exemplary command module of the patient monitoring system;

FIG. 7A is an oblique front view illustration of one embodiment of a docking station of the patient monitoring system;

FIG. 7B is side view illustration of one embodiment of a docking station of the patient monitoring system;

FIG. 7C is a rear view illustration a monitor and display assembly of the patient monitoring system docked to a docking station;

FIG. 8 is a block diagram illustration of an exemplary docking station printed circuit board assembly (PCBA);

FIG. 9A is an oblique side view illustration of one embodiment of a sidestream capnography or multigas pod of the patient monitoring system;

FIG. 9B is an oblique rear view illustration of one embodiment of the monitor and display assembly of the patient monitoring system depicting a sidestream capnography or multigas pod attached; and,

FIG. 10 is an oblique rear view illustration of one embodiment of the monitor and display assembly of the patient monitoring system depicting the monitor and display assembly docked in a docking station and a sidestream capnography or multigas pod attached.

### DETAILED DESCRIPTION

In one embodiment, the present specification is directed toward a configurable patient monitoring system comprised of a plurality of non-integrated components including a display and monitor assembly, optional additional stand-alone displays, optional additional stand-alone monitors, one or more modules, and at least one patient parameter measuring device. A variety of patient parameters can be monitored and the parameter measuring devices are connected to the system via Dual Serial Bus (DSB) connectors and DSB cables.

The DSB interface comprises a first serial protocol and a second serial protocol, wherein the first protocol is a Universal Serial Bus (USB), Firewire, or Ethernet protocol and the second serial protocol is a Low Power Serial (LPS) protocol. The DSB interface manages power distribution within the system by providing 5 V via the USB protocol or 3.3 V via the LPS protocol to connected devices. Within the DSB interface, each component of the patient monitoring system is a DSB Host, DSB Device, or, both a DSB Host and DSB Device. A DSB Host is in communication with and can supply operating and battery charging power to a connected DSB Device and additionally contains a switched Auxiliary Voltage Supply (AVS) which can provide up to 15 W of power to attached DSB Devices for battery charging or other high power needs. The DSB host recognizes the power requirements of the attached devices and switches power delivery accordingly. The DSB interface is presented in greater detail in co-pending U.S. patent application Ser. No. 13/300,478, entitled “Dual Serial Bus Interface”, filed on Nov. 18, 2011 and assigned to the Applicant of the present invention, which is hereby incorporated by reference.

### Monitor and Display Assembly

In one embodiment, the patient monitoring system includes a combined monitor and display assembly wherein the monitor is fixed securely and irremovably to the back of the display and the display is driven by the monitor.

In one embodiment, the monitor interfaces with the modules and allows for communication with external devices. The monitor is similar to a CPU tower and provides a dock for a parameter module and recorders. In one embodiment, the monitor contains one bay that provides power and communication for a proprietary Spacelabs module. In one embodiment, the monitor can support both current and old modules and also front end device (FED) patient parameter cables. In one embodiment, the monitor contains four USB ports to interface with devices including, but not limited to, keyboards, mice, bar code scanners, and thumb drives. A Patient Worn Hub (PWH), a small, portable self-contained monitor described in co-pending U.S. patent application Ser. No. 13/300,526 entitled “Self-Contained Patient Monitor”, filed on Nov. 18, 2011 and assigned to the Applicant of the present invention, which is hereby incorporated by reference, can also be connected. The PWH can also communicate wirelessly with the monitor. In these scenarios, the monitor acts as the DSB Host and the PWH is the DSB Device.

In addition, third party devices can be connected to the monitor via Device Interface Cables, which translate the output of the third party device to the protocol embedded within the DSB connector. The Device Interface Cable has a DSB connector at one end and a cable connector at the other end to interface with the host and the third party device respectively. The Device Interface Cable is described in greater detail along with the PWH in the application referenced directly above.

In one embodiment, the monitor contains a DVI port to allow for connection to an independent external display. The monitor also contains an Ethernet port for communication with other monitors and hospital infrastructures.

In one embodiment, the monitor contains an alarm relay output for an external nurse alert. This port is used for communication with an external display as described above. In one embodiment, one port is employed to carry both the signal to activate the alarm lights and the alarm audio, eliminating the need for two discrete cables and two discrete ports. In one embodiment, the monitor contains an additional nurse alert port that can be used with a stand-alone (not in a display) external nurse alert. The monitor also contains a Synchronous Data Link Control (SDLC) port for communication with expansion module bays that allows users to use more modules through one device. In one embodiment, the monitor contains a serial port for touch screen communication, software updates and data logging. In one embodiment, power is supplied to the monitor and display assembly via a DC power input. In one embodiment, the monitor and display assembly includes an equipotential terminal for grounding the monitor. The monitor and display assembly also contains a rechargeable battery that is used in the event of power interruption for back-up of module data, powering external nurse alerts, and powering the infrared (IR) receiver.

In one embodiment, the monitor utilizes “Smart” Li-Ion batteries to provide long battery life and safety. The design provides a custom form factor locking out insertion of incompatible batteries. A built in microcontroller monitors charging, discharging and over temp conditions. Thermal and current fuses are also provided as redundant safety features. An embodiment uses Inspired Energy NI2040HD24 Smart Battery including a rechargeable Lithium Ion battery and a Battery Management Module. The battery consists of 9 Lithium Ion rechargeable cells of 18650 size, assembled in a 3 series/3 parallel (3S 3P) configuration. Each cell has an average voltage of 3.6V and a typical capacity of 2.4 Ah giving a battery pack of 10.8V and 7.2 Ah. The battery is capable of communicating with host or the charger through a System Management Bus (SMBus). Protection is provided for over-charge, over-discharge and short circuit. For redundancy, passive safety devices are integrated into the pack to protect against over-current and over-temperature, and secondary over-voltage is implemented with a logic-fuse and controller.

In one embodiment, the monitor can run 8 hours on battery power while monitoring ECG, NIBP every 15 minutes and taking a recording every 15 minutes.

In one embodiment, the monitor utilizes Dynamic Network Access (DNA) to bring lab, pharmacy, charting, intranet, and Hospital Information System (HIS) applications to the bedside. Medical personnel are able to access this information using a Citrix thin client application running on the monitor. This requires a Citrix server to host the application to serve to the monitors. Nurses and physicians can review information from multiple sources without leaving the patient care area. Concise and complete electronic patient records are created effortlessly. In one embodiment, the monitor includes data shuffle and bar code scanner support for fast, error-free identification and transfer of patient information. With the DNA option, instant access to patient information is assured across the network. This results in assuring optimum patient safety while simultaneously maximizing caregiver efficiency. In one embodiment, the special feature Full Bed Review gives the nurse or physician the ability to remotely view, control, review, and record patient data for any other networked or telemetry bed without leaving the patient's bedside. In one embodiment, the special feature Remote View/Alarm Watch allows the caregiver to see any parameter for any monitored patient on the network from any bedside. During an alarm state, waveforms and numerical data may be saved and recorded for later review. In one embodiment, the special feature Alarm Limit Review provides the caregiver a snapshot view of bedside alarm limits for all active parameters for viewing or printing. In one embodiment, the special feature ICS Clinical Event Interface instantaneously transmits alarms and waveforms to personal communication devices for immediate viewing, resulting in quicker response times. In one embodiment, flexport interfaces link patient data from standalone devices, consolidating waveforms, data, and alarms within the monitor. Information is then integrated directly into the monitor trends for output to HIS and CIS applications.

In one embodiment, the monitor and display assembly includes a pod connection port for the addition of a capnography or multigas pod as described below.

In one embodiment, the circuit boards of the monitor are all mounted onto a removable internal chassis. The chassis can be removed from the enclosure while still keeping the monitor fully functional. Each circuit board is individually accessible to allow service personnel to easily troubleshoot all circuit boards/components and replace any one board/component without having to completely disassemble the monitor.

In one embodiment, the monitor and display assembly includes a printer slot to expand the capabilities of the monitor. In one embodiment, the printer accepts 50 mm paper.

In one embodiment, the monitor and display assembly includes a handle that can rotate to up and down positions. When the handle is released or let go of, it gradually drops to its default down position. In one embodiment, a rotational damper retards the downward motion of the handle (on release, from its up position) so that the handle does not slam into the monitor and display assembly. This allows for quiet use of the handle and monitor and display assembly without disturbing patients. In accordance with an embodiment, the handle also has a functionality to stop at a predetermined set-point at which the monitor and display assembly is balanced enabling the display to be perpendicular to the floor when being carried using the handle. This functionality allows the monitor and display assembly to be quite comfortable to hold and walk with, using the handle, as it does not get in the way of the user's leg nor puts awkward forces on her arm/hand.

In one embodiment, Sabic Lexan EXL plastic is utilized for monitor and display assembly housing to allow the monitor and display assembly to withstand an unintentional drop, chemical cleaning and excessive heat. In one embodiment, the monitor and display assembly weighs less than 9 pounds.

In one embodiment, the display includes a 12.1 inch touchscreen and is capable of depicting up to eight waveforms. In one embodiment, the monitor and display assembly contains speakers for audio alarms.

In one embodiment, the external display contains integrated visual alarm lights located on the front and back of the monitor and display assembly. These alarm lights are larger than current visual alarms, providing a better visual indicator to medical personnel during alarm situations. In one embodiment, the alarm lights flash red, yellow, and cyan to indicate high, medium, and low priority alarms respectively. The alarm lights are configurable by a user to define the minimum level of alarm lights that may be activated independent of the on screen alarm display and/or alarm audio. A continuous, flat piece of glass occupies the entire front of the display and sets into a metal band that wraps the exterior sides of the display, as a frame, for robustness. The piece of glass contains no bezels and doubles as both a touchscreen and as the lens and means of light dispersion for the visual alarm, resulting in a reduced part count. The flat touchscreen glass also provides a continuous surface presented at the front. This makes cleaning easier as there are no edges as found in typical bezel implementations which provide crevices for accumulation of contaminants. In one embodiment, the metal band extends slightly out past the touchscreen to protect the glass if dropped on its face. It should be appreciated by those of ordinary skill in the art that the metal band/frame with the flat bezeless touch screen gives a contemporary look to the monitor and display assembly. In other words, the monitor and display assembly looks akin to consumer electronics such as flat screen TVs and cell phones. This can help ease and acclimatize the patient and patient's family since the display looks more like a home electronic device and familiar. The monitor and display assembly also has soft edges as part of the design to make it look less industrial and more friendly and approachable.

A light source behind the glass transmits appropriate wavelengths of light to indicate alarms. In one embodiment, a black border is silk-screened on the back of the glass around the perimeter. In one embodiment, the black border is comprised of an ink that is silk-screened or sprayed onto a masked out border area that gives the appearance of a continuous and uniform black border but allows light to pass through when the alarm is activated, yielding a visual alarm.

In another embodiment, the border area that is used for the visual alarm contains small apertures that make the border appear continuous and uniform but allow light to pass through. This provides a clean, flat modern appearance that shows no indication of alarm until an actual alarm occurs.

In one embodiment, the nurse alarm signals, including flash rates for the display and audio for the audible alarms, are driven and controlled by the monitor.

In one embodiment, the external display contains an ambient light sensor that senses the brightness level of the environment and adjusts the display brightness accordingly. In a darker or poorly lit environment, the ambient light sensor will automatically dim the display and the alarm lights. This is particularly beneficial for instances in which the patient is sleeping, as dimmer lights will be less likely to disturb the patient. In a brighter or well-lit environment, the ambient light sensor will automatically brighten the display. This feature can be deactivated by a button on the display.

In one embodiment, the external display contains a capacitive button on the front of the touchscreen that can be programmed by the user to perform a variety of functions. In various embodiments, the button is a metal plate or other conductive material utilizing any commonly used touch and/or pressure sensitive technologies. The button is large and positioned prominently as compared to a smaller touch screen button so that it can be accessed easily. In one embodiment, the button is located on the top edge of the front of the display. In another embodiment, the button is located on the bottom edge of the display. In another embodiment, the button is located on the left edge of the display. In another embodiment, the button is located on the right edge of the display. In addition, the button is easier to find because it is not obscured by the clutter of other buttons or user interface items. Circuitry in the monitor senses when the button is touched by an operator and the monitor executes the programmed function.

In one embodiment, the button is programmed to suspend alarm when touched. This allows medical personnel to quickly silence an alarm and reset the alarm indications, so that they can tend to the patient's needs and prevent disturbance to other patients in the area. Since alarms are produced in response to critical events, it is important that the means for silencing and/or resetting them be easy to find and quick to activate. In another embodiment, the button is programmed to admit patient when touched. In another embodiment, the button is programmed to initiate NIBP measurement when touched. In another embodiment, the button is programmed to return the display to its home screen when touched. In yet another embodiment, the button is programmed to print the display when touched. The button would primarily be programmed to suspend alarm to simplify the action required by a nurse to silence an alarm. However, one skilled in the art will understand that the button could be programmed to perform a variety of functions not limited to those listed above.

In another embodiment, the display contains includes a section of the touchscreen programmed for control of the alarm light.

In one embodiment, the external display contains a back-lit power button on the side with a power symbol that is green when the monitor and display assembly is switched on.

The display is housed with a metal band and a powder coated finish. The back of the monitor and display assembly contains a mounting pattern for standard 75 mm Video Electronics Standards Association (VESA) mounts.

### External Display and Alarm Indicators

In one embodiment, the patient monitoring system includes one or more optional stand-alone displays as disclosed in U.S. patent application Ser. No. 13/300,462, entitled “Configurable Patient Monitoring System”, filed on Nov. 18, 2011 and assigned to the applicant of the present invention, which claims priority from U.S. Provisional Patent Application No. 61/415,799, entitled “Patient Monitoring System with Dual Serial Bus (DSB) Interface” and filed on Nov. 19, 2010, which are both herein incorporated by reference in their entirety.

### Monitor

In one embodiment, the patient monitoring system includes one or more optional stand-alone monitors as disclosed in U.S. patent application Ser. No. 13/300,462, entitled “Configurable Patient Monitoring System”, filed on Nov. 18, 2011 and assigned to the applicant of the present invention, which claims priority from U.S. Provisional Patent Application No. 61/415,799, entitled “Patient Monitoring System with Dual Serial Bus (DSB) Interface” and filed on Nov. 19, 2010, which are both herein incorporated by reference in their entirety.

### Docking Station

In one embodiment the monitor and display assembly is enabled for portability using a docking station that provides a quick single button press un-docking of the monitor and display assembly therefrom while still maintaining patient monitoring for transport/emergency scenarios. The docking station allows flexibility and ease of use of the monitor and display assembly with respect to connect and disconnect from power, Ethernet, external display and other external patient parameter measuring devices. In one embodiment, receptacles such as Ethernet connection, DVI for external display, USB, serial ports, external nurse alert/external audio/IR receiver, power and SDLC (synchronous data link control) port are duplicated on the docking station. Further, the docking station allows for greater portability of the patient monitoring system in the hospital environment, where space is often limited and cluttered with other medical equipment. In one embodiment of the present invention, the patient cables are always attached to the patient, both when the monitor and display assembly is docked and undocked. Thus, the cables do not need to be removed from the patient or the monitor and display assembly and stay connected to the patient so that the patient can be continuously monitored.

In one embodiment, all external signals are routed to a single docking connector located on the bottom of the monitor and display assembly and mating connector at the top of the docking station. These signals are switched active when the monitor and display assembly is docked and remain inactive when un-docked so that voltages are absent on the mating connector pins when the monitor and display assembly is not docked (to prevent accidental electric shock to users when the connector pins are exposed in a not docked scenario).

In one embodiment, the docking station is structurally contoured, along with a standard 4-hole VESA mounting pattern duplication thereon, to allow the same external wall, roll stand and fixed mounts used with the monitor and display assembly to work with the docking station. In one embodiment, a contoured feature on the back of the docking station is designed to cover the plurality of receptacles/ports on the back of the monitor and display assembly, when docked, so that the receptacles are prevented from being connected more than once. The contoured feature also comprises venting to allow the monitor and display assembly intake vents at the bottom to remain unobstructed when docked.

In one embodiment, the docking station has a molded recess around the edge of the perimeter that matches the monitor and display assembly's external shape around the bottom of the monitor and display assembly. This molded recess on the dock has a slight bevel outwards that helps guide the monitor and display assembly into position as a first coarse adjustment. In one embodiment, two large domed guide pins engage upon further placement of the monitor and display assembly into the dock and settle the monitor and display assembly exactly, smoothly mating the monitor and display assembly and docking station connectors.

In one embodiment, the docking station has a prominent button in the front that disconnects the latching and is used for undocking the monitor and display assembly. In one embodiment, the docking station button is backlit when the monitor and display assembly is docked to allow easy recognition of its location in a darkened room.

### Module

The patient monitoring system of the present invention also includes a module which provides measurements of a plurality of patient parameters. Many types of modules exist and can be utilized, depending on which patient parameters are needed.

In one embodiment, the patient monitoring system includes a command module. The command module can measure both adult and neonatal NIBP, IBP, ECG, SpO₂, cardiac output, and temperature and includes a stop button to manually override NIBP measurements. The command module communicates via Synchronous Data Link Control (SDLC) bus with and derives power from the patient monitor. In addition, the command module contains internal memory to allow the module to be taken with a patient during transport and plugged into a separate monitor and display assembly or stand-alone monitor without losing data. In one embodiment, the command module is the core of the patient monitoring system, providing the processing power for all basic physiologic parameters. Caregivers are able to select from a variety of configurations to suit the monitoring needs of specific patients or care units in the hospital. In another embodiment, the command module includes three levels of arrhythmia monitoring (basic, standard multi-view, and advanced multi-view) as well as diagnostic 12-lead ECG analysis and reports with or without measurement and interpretation. In addition, the command module also includes ST-segment analysis and event review or Varitrend 4 for event review of neonatal respiration, heart rate, and SpO₂.

In one embodiment, the patient monitoring system includes a capnography module which measures the end tidal CO₂, minimum inspired CO₂, and respiratory rate to aid in evaluating the respiratory status of any adult, child, or infant patient. Routine calibrations are not required because the module automatically compensates for ambient barometric pressure. In one embodiment, the capnography module is flexible in that it combines both mainstream and sidestream monitoring modes in a single unit. Sidestream monitoring includes a low sampling rate of 50 ml/min which is ideal for smaller patients. In addition, the capnography module enables the user to obtain waveform data, numeric values (kPa, mm Hg, or %), minimum inspired CO₂ values, and airway respiration rates. This data can further be displayed, incorporated into trends, and/or output to charting applications.

In one embodiment, the patient monitoring system includes a Bispectral Index (BISx) module which measures depth of consciousness and sedation level of patients in operating room and critical care environments, eliminating the need for bulky standalone systems. This type of module is used to prevent patients' awareness during surgery by notifying clinicians when additional medication is needed. The BISx analysis is calculated from the frequency, power, and phase throughout the entire frequency range of the EEG and presented as an index number between 1 and 100. Adult and pediatric sensors work with the same module, which is easily moved from one monitor to another.

In one embodiment, the patient monitoring system includes a mixed Venous Oxygen Saturation (SvO₂) module which measures SvO₂ and Central Venous Oxygen Saturation (ScvO₂) to assess the balance of oxygen delivery and consumption. Venous oxygen saturation is being increasingly used in critically ill patients, often as part of an early goal-directed therapy protocol and in sepsis screening to aid in the assessment of cardiovascular and respiratory compromise. Catheter placement in venous monitoring is less invasive than in arterial monitoring, making it available to more patients. The ScvO₂ probe may be placed into an existing 16 cm or 20 cm central line, reducing or eliminating the need to exchange central venous catheters in order to provide continuous ScvO₂ monitoring.

In one embodiment, the patient monitoring system includes an EEG module which measures and displays brainwave activity. In one embodiment, this module also includes one channel of electromyogram (EMG) monitoring, measuring and displaying muscle electrical activity. Data storage options include two, eight, or 24 hours or snapshots. The data can be displayed as an analog moving waveform or as a density spectral array (DSA). A number of trends are available, including magnitude trends, power ratio trends, and a selection of frequency trends. Integrated electrosurgical protection assures patient safety. In one embodiment, the module is enclosed by two pieces of sheet metal.

### Capnography/Multigas Pod

In an embodiment, the patient monitoring system includes an externally mounted sidestream capnography or multigas pod attachable to the rear of the monitor and display assembly. Therefore, capnography or multigas functions can be added to any monitor display assembly that is configured to accept such an externally mountable pod. In one embodiment, the pod receives power from the monitor and display assembly and communicates through pogo pins. Large guide pins at the bottom of the pod allow it to be blind mated to the rear of the monitor and display assembly. In accordance with an embodiment, the guide pins have ball stud ends that provide a positive lock and retention force to the monitor and display assembly when fully engaged. The monitor has gold immersion contact pads to allow power, ground and signal contacts that are recessed with a small diameter so the user cannot touch live voltages present on the contact pins. In an embodiment, a push button on the pod provides a mechanical actuation of a latching mechanism for connecting and removing the pod from the monitor and display assembly. Persons of ordinary skill in the art would appreciate that the modular configuration of the pod allows users to selectively outfit monitor and display assemblies with either capnography or multi-gas based on need.

The present invention is directed toward multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be appreciated that electronic communication between devices may be effectuated by the transmission and receipt of data between applications executing in any of the devices or computing systems. Each application is configured to receive, transmit, recognize, interpret, and process such request data and information. It should further be appreciated that both the system described herein have receivers and transmitters capable of sending and transmitting data, at least one processor capable of processing programmatic instructions, memory capable of storing programmatic instructions, and software comprised of a plurality of programmatic instructions for performing the processes described herein.

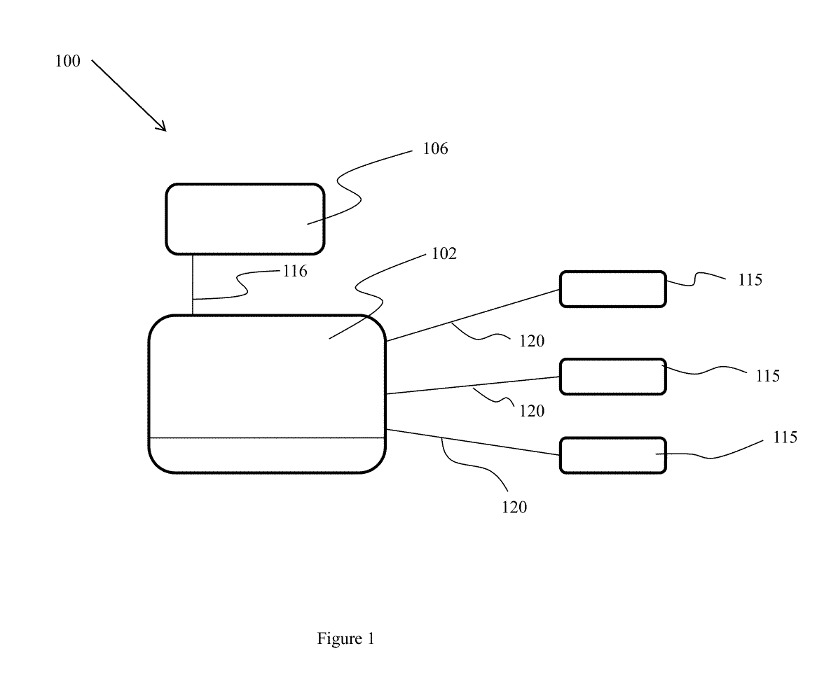

FIG. 1 is a block diagram depicting one embodiment of an exemplary configuration of the components of the patient monitoring system 100, illustrating the use of DSB cables 120 to connect patient parameter measuring devices 115 to the monitor and display assembly 102. In this embodiment, one module 106 is connected to the monitor 102 via a DSB cable 116, which is directly connected to a DSB connector in the module bay (not shown).

Patients are often transported between care areas of the hospital. It is common practice to provide monitoring of parameters such as ECG, SpO2, NIBP, capnography and other parameters even during transport, especially for critically ill patients. Therefore, in accordance with an aspect of the present specification, the patient monitoring system comprises a combined monitor and display assembly enabled for portability and overall compactness. The monitor and display assembly uses a docking station that is configured for a single button-press docking and un-docking of the monitor and display assembly. Also, in one embodiment, an externally mountable capnography or multigas pod is attachable to the rear of the docked or undocked monitor thereby providing overall modularity of design for portability.

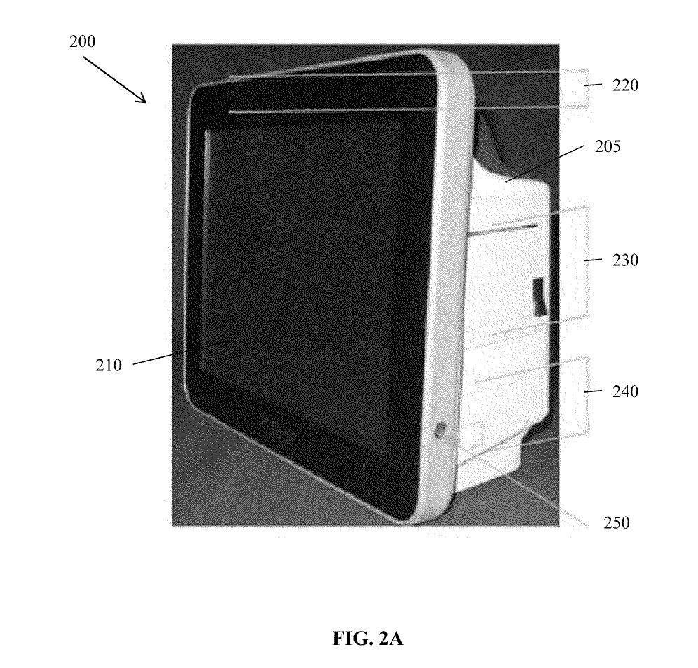

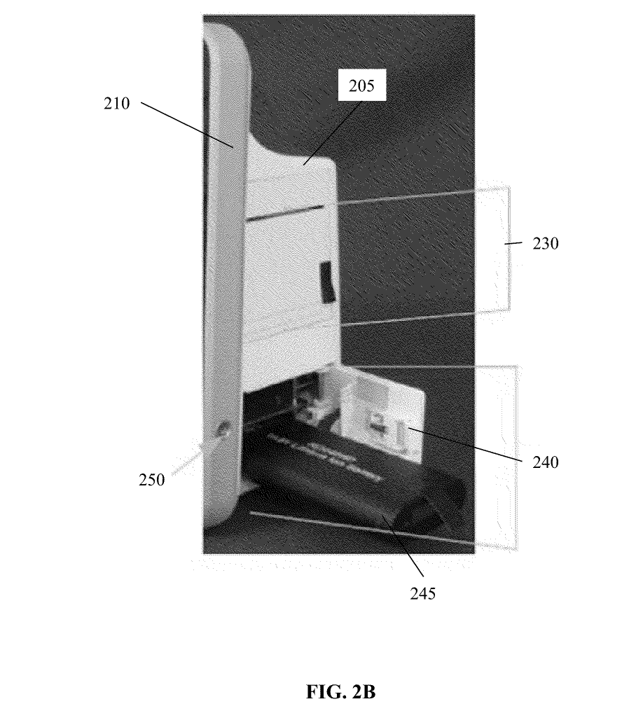

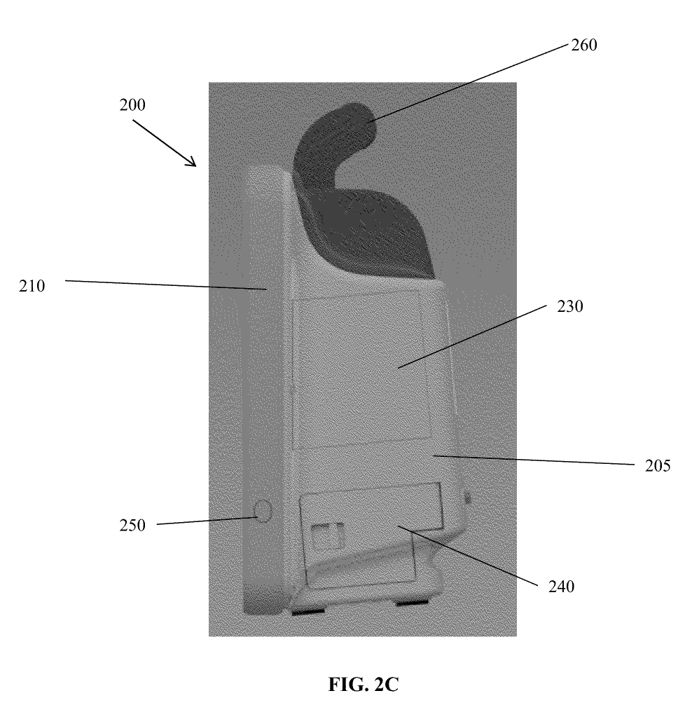

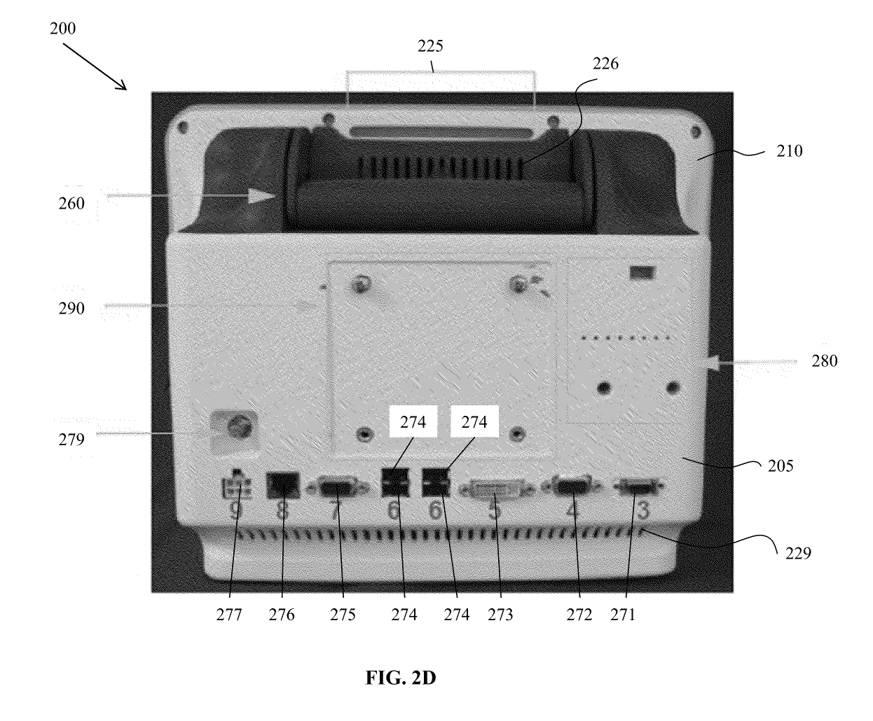

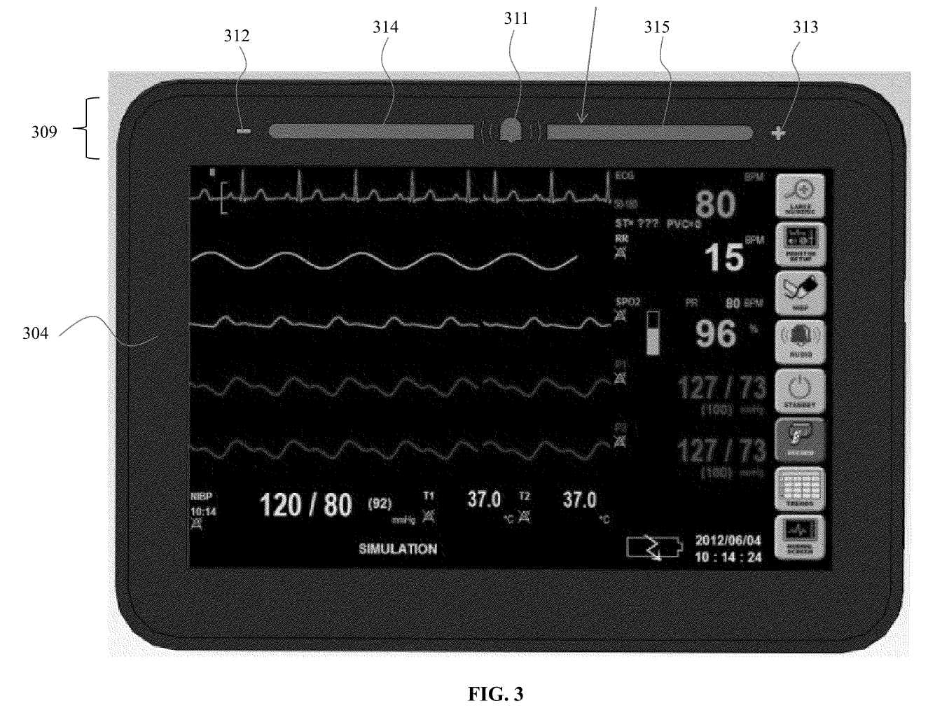

FIG. 2A is an oblique side view illustration of one embodiment of a monitor and display assembly 200 of the patient monitoring system. The assembly 200 includes a monitor 205 fixed operatively and irremovably to the back of a display 210. In one embodiment, the display 210 includes a front alarm area 220 proximate the top edge of the front of the display 210. In one embodiment, the front alarm area 220 also functions as a touchscreen allowing user control of alarm volume, as discussed with reference to FIG. 3. The monitor and display assembly 200 also includes a power button 250. In the pictured embodiment, the power button 250 is positioned on the lower right side of the display 210. In one embodiment, a user can power on or off the assembly 200 by pressing down the power button 250 for 3 seconds. In one embodiment, the power button 250 is illuminated with a green backlight to indicate on status. In one embodiment, the assembly 200 includes a progress bar (not shown) just below the power button 250. The progress bar fills to indicate the start up process to the user.

In one embodiment, the monitor 205 of the monitor and display assembly 200 includes a printer slot 230 for the addition of a printer to expand the capabilities of the monitor and display assembly 200. In one embodiment, the printer accepts 50 mm paper. In one embodiment, the monitor 205 of the monitor and display assembly 200 includes a battery compartment cover 240 that covers the rechargeable battery compartment.

FIG. 2B is an oblique side view illustration of one embodiment of the monitor 205 and a portion of the display 210 of a monitor and display assembly 200, depicting a rechargeable battery 245 partially removed from the monitor 205. The power button 250 is positioned on the lower right side of the display 210. In the pictured embodiment, the battery compartment cover 240 has been opened and the battery 245 has been partially slid out of the monitor 205. In one embodiment, the battery 245 comprises “Smart” Li-Ion batteries as described above.

FIG. 2C is a side view of an embodiment of a monitor and display assembly 200 illustrating a handle 260 in an up position. In one embodiment, the monitor and display assembly 200 comprises a monitor 205 fixedly attached, and in communication with, a display 210. In one embodiment, the monitor 205 is fixedly attached to the back of the display 210 via a set of screws. The handle 260, in one embodiment, can rotate to up and down positions. When the handle 260 is released or let go of, it gradually drops to its default down position (not shown). In one embodiment, a rotational damper retards the downward motion of the handle 260 (on release, from its up position) so that the handle 260 does not slam into the monitor 205. This allows for quiet use of the handle 260 and monitor and display assembly 200 without disturbing patients. In accordance with an embodiment, the handle 260 has a predetermined set-point at which the monitor and display assembly 200 is balanced enabling the display 210 to be perpendicular to the floor when being carried using the handle 260. This allows the monitor and display assembly 200 to be quite comfortable to hold and walk with, using the handle 260, as it does not get in the way of the user's leg nor put awkward forces on her arm/hand.

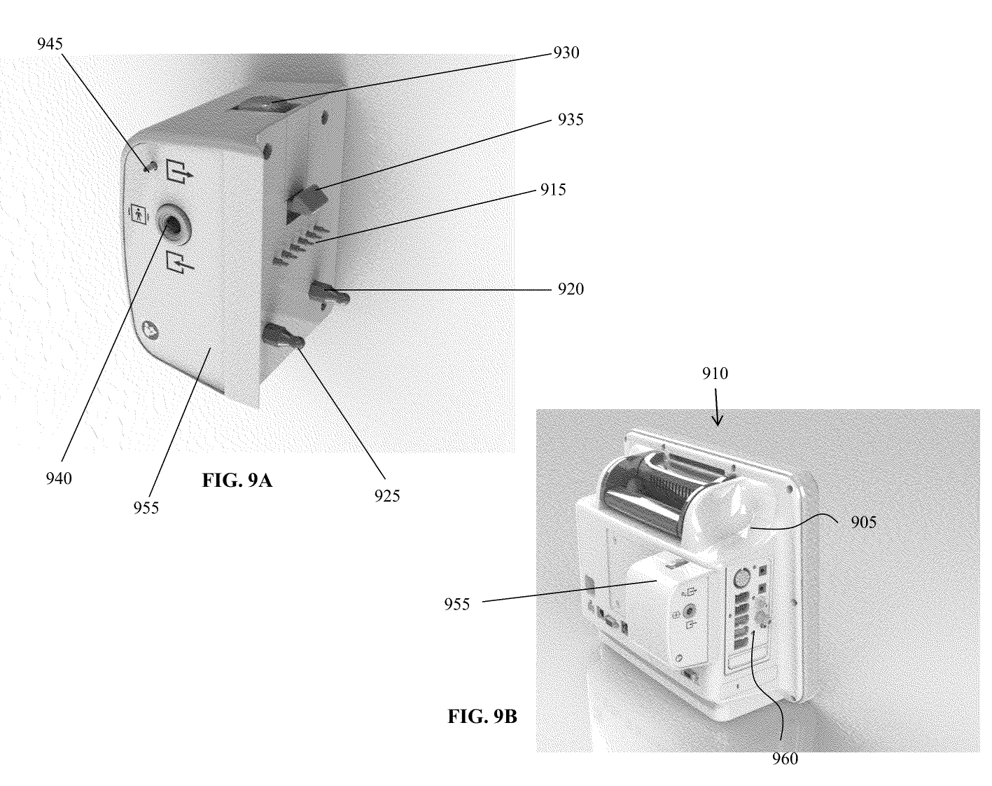

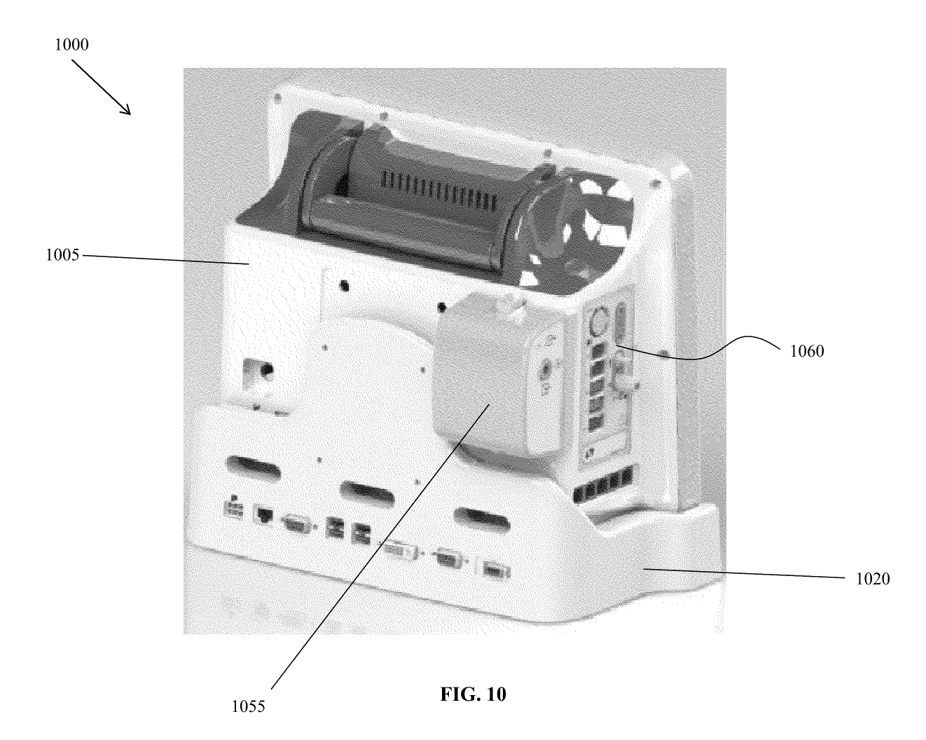

In the pictured embodiment, FIG. 2C shows the right side of the monitor and display assembly 200 and also depicts the power button 250, printer slot 230, and battery compartment cover 240. The left side of the monitor and display assembly 200 includes a slot for inserting a module (shown in FIGS. 9B and 10).

FIG. 2D is a rear view illustration of one embodiment of a monitor and display assembly 200 depicting a plurality of receptacles. The display 210 portion includes a back alarm light 225 to allow for visibility of visual alarms from the back of the assembly 200. The assembly includes a set of intake vents 226, 229 at the top and bottom of the monitor 205. The back of the monitor 205 includes standard 75 mm VESA mounting holes 290 for mounting of the assembly 200. In one embodiment, the monitor 205 also includes a connection port 280 for a capnography or multigas pod as described with reference to FIGS. 9A, 9B, and 10. In one embodiment, the assembly 200 includes an equipotential terminal 279 for grounding the monitor 205.

In one embodiment, the assembly 200 includes a plurality of receptacles across the lower back surface of the monitor 205. In various embodiments, these receptacles include an alarm relay output for nurse alert 271, an SDLC port 272, a DVI port for video output 273, 4 USB ports 274, a serial port 275, an Ethernet port 276, and an input port for DC power 277.

In one embodiment, the display includes an area of the touchscreen for alarm volume control. FIG. 3 is a front view illustration of another embodiment of the patient monitoring system depicting an external display 304 with a red alarm light 310 on the front of the display 304. In the pictured embodiment, the glass is treated such that it allows the transmitted light to pass through. A black border is silk-screened on the back of the glass around the perimeter. The black border is comprised of an ink that is silk-screened or sprayed onto a masked out border area that gives the appearance of a continuous and uniform black border but allows light to pass through when the alarm is sounded, yielding a visual alarm. Thus, the black border of the display 304 appears uniform and continuous until an alarm occurs. Once an alarm is activated, a light source built into the body of the display 304 transmits light in an appropriate wavelength to the glass covering the front of the display 304 to indicate alarms. In another embodiment, the glass contains small apertures that allow the transmitted light to pass through. The display 304 includes an active touchscreen area 309 proximate the top that allows for alarm volume control. In one embodiment, during an active alarm state, a visual alarm bar 310 is illuminated proximate the top of the display 304. In one embodiment, the visual alarm bar 310 flashes during an active alarm state. In another embodiment, the visual alarm bar 310 remains solidly illuminated during an active alarm state. In one embodiment, the illumination is provided by LEDs behind the glass. In one embodiment, a red light signifies a high priority alarm. The display is also capable of transmitting a yellow light signifying a medium priority alarm and a cyan light signifying a low priority alarm. The alarm lights are configurable by a user to define the minimum level of alarm lights that may be activated independent of the on screen alarm display and/or alarm audio.

In one embodiment, the visual alarm bar 310 includes a bell shaped icon with emanating sound waves 311 (as pictured) or any other similar icon used to notify medical personnel of an alarm state. The bell icon 311 is positioned in the center of the visual alarm bar 310. The visual alarm bar 310 further includes a minus icon 312 with a decrease bar 314 between the minus icon 312 and the bell icon 311 positioned on one side of the bell icon 311 and a plus icon 313 with an increase bar 315 between the plus icon 313 and the bell icon 311 positioned on the opposite side of the bell icon 311. In various other embodiments, the minus icon can be any other icon that conveys a meaning of decrease, such as a down pointing arrow, and the plus icon can be any other icon that conveys a meaning of increase, such as an up pointing arrow. In the pictured embodiment, the minus icon 312 is positioned to the left of the bell icon 311 and the plus icon 313 is positioned to the right of the bell icon 311. In another embodiment, the icon positions are reversed. In one embodiment, the minus icon 312 and plus icon 313 are illuminated green. In one embodiment, the minus icon 312 and plus icon 313 illuminate when the visual alarm bar 310, including the bell icon 311, decrease bar 314, and increase bar 315, is illuminated (in other words, when an active alarm state begins). In another embodiment, only the bell icon 311, decrease bar 314, and increase bar 315 components of the visual alarm bar 310 illuminate when an active alarm begins and a user must press anywhere on the visual alarm touchscreen area 309 for the minus icon 312 and plus icon 313 to appear, allowing volume control.

When there is no alarm active, the visual alarm bar 310 appears blacked out. During an active alarm state, the visual alarm bar 310 is illuminated a specific color corresponding to the current alarm level. A user can decrease the alarm volume by pressing anywhere in the touchscreen area 309 that is on the minus icon 312, the decrease bar 314, or on the part of the bell icon 311 that is on the same side as the minus icon 312. A user can continue to decrease the alarm volume by repeatedly pressing said area. A user can increase the alarm volume by pressing anywhere in the touchscreen area 309 that is on the plus icon 313, the increase bar 315, or on the part of the bell icon 311 that is on the same side as the plus icon 313. A user can continue to increase the alarm volume by repeatedly pressing said area.

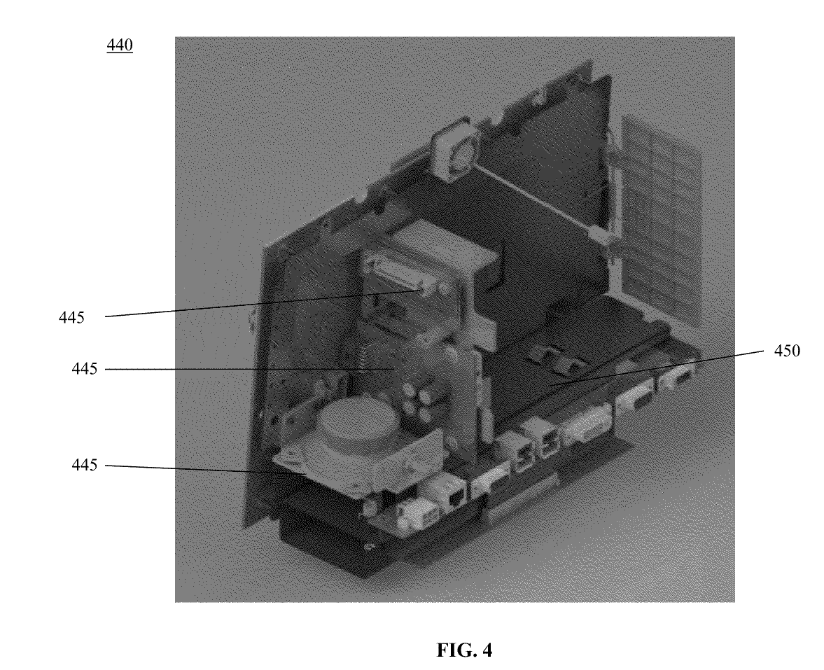

FIG. 4 is an oblique inside-view illustration of an embodiment of the internal components of a monitor 440 of a monitor and display assembly wherein circuit boards 445 of the monitor 440 are all mounted onto a removable internal chassis 450. The chassis 450 can be removed from the monitor and display assembly enclosure while still keeping the monitor 440 fully functional. Each circuit board 445 is individually accessible to allow service personnel to easily troubleshoot all circuit boards/components and replace any one board/component without having to completely disassemble the monitor 440.

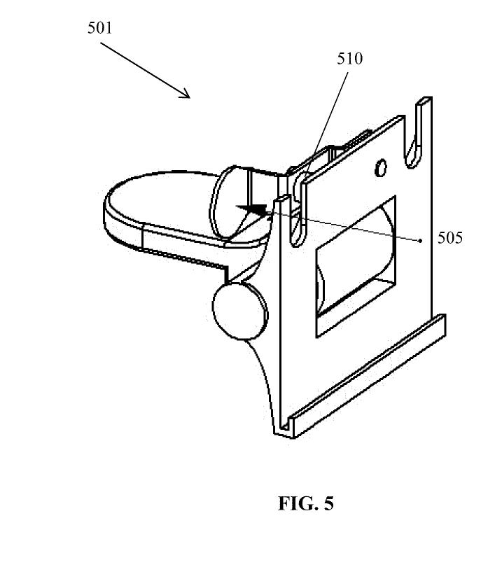

FIG. 5 is an oblique front view illustration of one embodiment of a quick release mount 501 that allows quick disengagement of a monitor and display assembly from a fixed mount—such as those on a wall, anesthesia machine, table top, etc. Lever 505 slides over pin 510 and allows finger pressure to release the pin. This allows easy disengagement of mounts from the front of the monitor and display assembly.

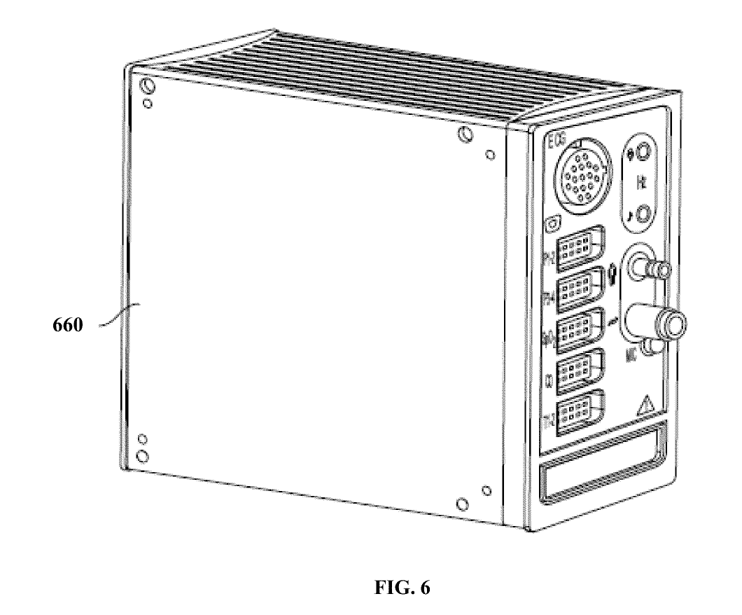

FIG. 6 is an oblique front view illustration of one embodiment of a command module 660 of the patient monitoring system. In one embodiment, the command module can measure both adult and neonatal NIBP, IBP, ECG, SpO₂, cardiac output, and temperature and includes a stop button to manually override NIBP measurements. In one embodiment, the command module communicates via SDLC bus with and derives power from Spacelabs Healthcare monitors. In one embodiment, the command module contains internal memory to allow the module to be taken with a patient during transport and plugged into a separate monitor without losing data. In one embodiment, the module is enclosed by two pieces of sheet metal. In one embodiment, the module measures 2.2 inches wide by 4.5 inches high×7.0 inches thick. In other embodiments, the module measures from 1.9 to 2.5 inches wide×3.5 to 5.5 inches high×5.0 to 9.0 inches thick.

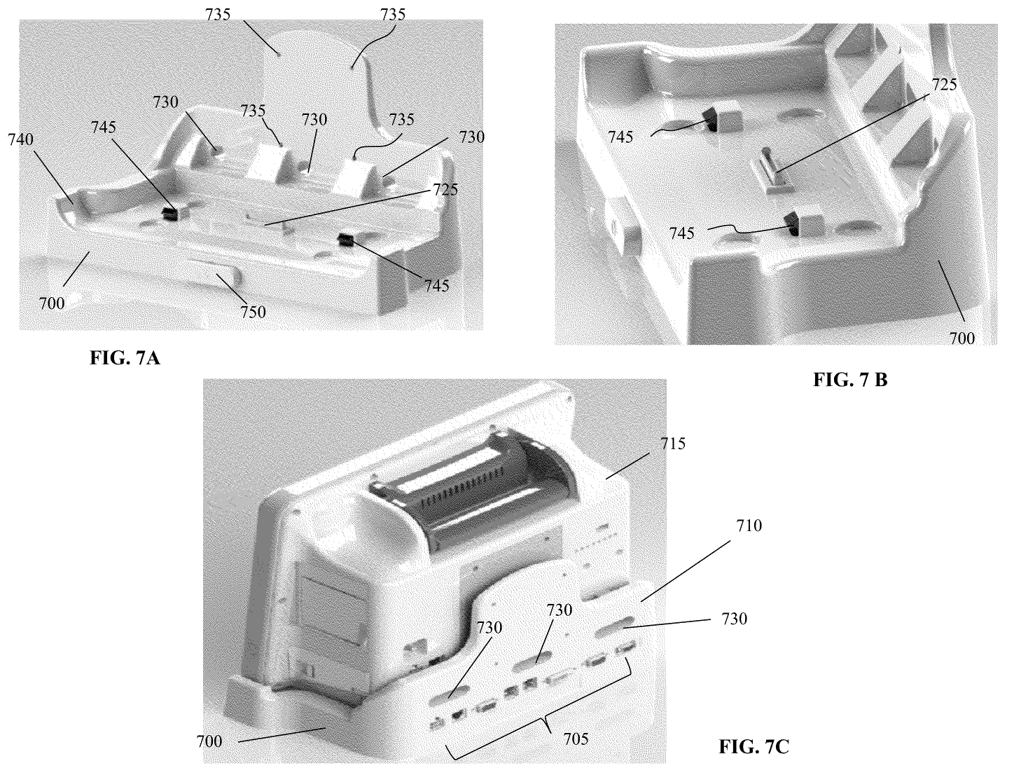

FIGS. 7A and 7B are different front view illustrations of an embodiment of the docking station 700 that allows single button press un-docking of a monitor and display assembly. FIG. 7C shows a rear view illustration of the monitor and display assembly 715 docked to the station 700. Referring now to FIGS. 7A through 7C simultaneously, a plurality of receptacles 705 such as Ethernet connection, DVI for external display, USB, serial ports, external nurse alert/external audio/IR receiver, power and SDLC (synchronous data link control) port are replicated on the docking station 700. A contoured feature 710 covers the receptacles on the back of the monitor and display assembly 715, when docked, so that the receptacles are prevented from being connected more than once. All external signals are routed to a single docking connector (not visible in the figures) located on the bottom of the monitor and display assembly 715 and mating connector 725 on the receiving surface of the docking station 700. These signals are switched active when the monitor is docked and remain inactive when un-docked so that voltages are absent on the mating connector pins 725 when the monitor and display assembly 715 is not docked.

In accordance with an embodiment, a plurality of vents 730, in the contoured feature 710, allow monitor and display assembly 715 intake vents at the bottom to remain unobstructed when docked. In one embodiment, the docking station 700 is structurally contoured, along with a standard 4-hole VESA mounting pattern 735 duplication, to allow the same external wall, roll stand and fixed mounts used with the monitor and display assembly 715 to work with the docking station 700. In one embodiment, a molded recess 740 around the edge of the perimeter of the docking station 700 matches the monitor and display assembly's 715 external shape around the bottom of the monitor and display assembly 715. The molded recess 740 on the docking station 700 has a slight bevel outwards that helps guide the monitor and display assembly 715 into position as a first coarse adjustment. In one embodiment, two large domed guide pins 745 engage upon further placement of the monitor and display assembly 715 into the dock 700. The guide pins 745 help settle the monitor and display assembly 715 exactly and smoothly mate the monitor and display assembly connector with the docking station connector 725.

An embodiment of the docking station 700 comprises a button 750 in the front that disconnects the latching and is used for undocking the monitor and display assembly 715. Optionally, the button 750 is backlit when the monitor and display assembly 715 is docked to allow easy recognition of location in a darkened room.

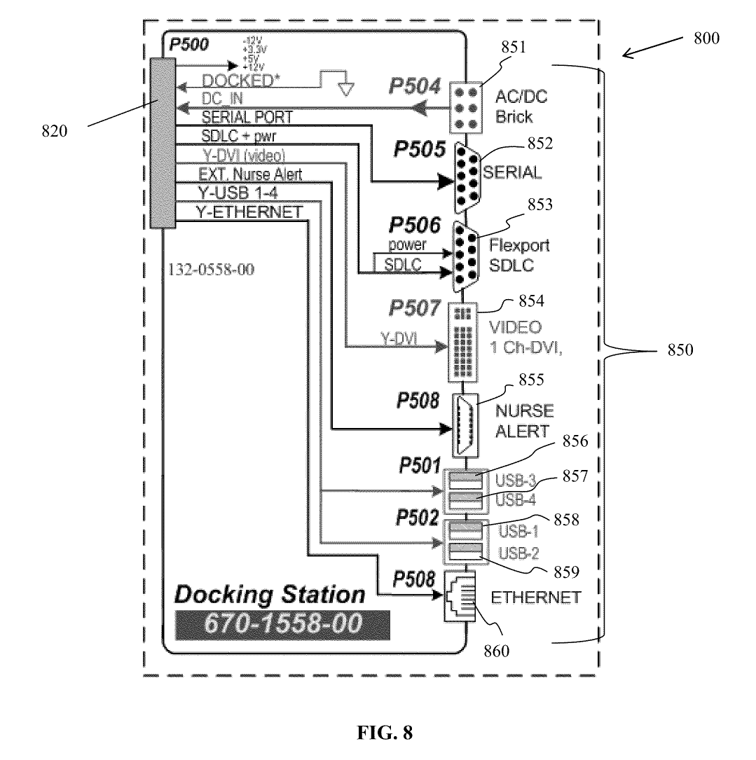

FIG. 8 shows a block diagram illustration of an exemplary docking station PCBA (Printed Circuit Board Assembly) 800 where connector 820 mates with a corresponding connector on the bottom of the monitor and display assembly, when docked, and routes all external signals from the monitor and display assembly to the plurality of receptacles 850 replicated on the docking station. In one embodiment, an AC/DC Brick receptacle 851 transfers DC power in to the monitor and display assembly via connector 820. In one embodiment, connector 820 provides serial communication to the monitor and display assembly via serial port 852. In one embodiment, SDLC and power are communicated from connector 820 to an SDLC Flexport 853. In one embodiment, a Y-DVI video signal is communicated from connector 820 to a 1 Channel DVI Video port 854. In one embodiment, external nurse alert information is communicated from connector 820 to a nurse alert port 855. In one embodiment, four Y-USB signals are communicated from connector 820 to two separate four USB ports 856, 857, 858, 859. In one embodiment, a Y-Ethernet signal is communicated from connector 820 to an Ethernet port 860.

FIGS. 9A and 9B show an embodiment of a sidestream capnography or multigas pod 955 that is externally mountable for attaching to the rear of the monitor and display assembly 900. In one embodiment, the pod 955 receives power from the monitor and display assembly 900 and also communicates through a plurality of pogo pins 915. The pins 915 provide a larger area for grounding contact, allowing less pogo pins to be required for mating connectors provided at the rear of the monitor and display assembly. Large guide pins 920 at the bottom of the pod allow it to be blind mated to the rear of the monitor and display assembly 900. In accordance with an embodiment, the guide pins 920 have ball stud ends 925 that provide a positive lock and retention force to the monitor and display assembly when fully engaged. In accordance with an embodiment, the monitor and display assembly 900 has gold immersion contact pads to allow for power, ground and signal contacts. The contact pads are incorporated within small diameter recesses so that users cannot touch live voltages present on the contact pins. In an embodiment, a push button 930 on the pod provides a mechanical actuation of a latching mechanism 935 for connecting and removing the pod 955 from the monitor and display assembly 900. Receptacles, such as inlet port 940 and scavenging port 945 are provided on one side of the pod 955. Persons of ordinary skill in the art would appreciate that the modular configuration of the pod 955 allows users to selectively outfit monitor and display assemblies with either capnography or multi-gas based on need.

FIG. 9B also depicts a module 960 inserted into the left side of the monitor 905 of the monitor and display assembly 900.

FIG. 10 is a rear view of the monitor and display assembly 1000 docked into the docking station 1020, for portability, and also having an externally mounted capnography or multigas pod 1055, in accordance with an embodiment. FIG. 10 also depicts a module 1060 inserted into the left side of the monitor 1005 of the monitor and display assembly 1000.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A system for patient monitoring comprising:
at least one patient monitor that allows for communication with external devices, wherein said patient monitor is in electronic communication with and drives at least one display, and wherein said display comprises: a housing having a front and a back; a touchscreen mounted to the front of said housing, wherein said touchscreen comprises a flat piece of glass having a central display area and a black border that extends along a left, right, top, and bottom edge of said glass, further wherein said monitor is fixedly attached to said back of said display;
at least one module for providing measurements of a plurality of patient parameters, wherein said module is in electronic communication with said patient monitor and wherein said module comprises at least one interface for electronically communicating with at least one patient parameter measurement device;
a processor for determining an alarm state; and, light sources within said touchscreen which are activated by said processor during the alarm state, wherein said light sources are configured to pass through said black border; and
at least one Dual Serial Bus (DSB) interface for enabling electronic communication between the patient monitor, module, and/or patient parameter measuring device.

2. The system for patient monitoring of claim 1, wherein at least one light source is positioned proximate said top edge of said front face of said display device.

3. The system for patient monitoring of claim 2, wherein said touchscreen comprises an area corresponding to said light source for controlling alarm volume level.

4. The system for patient monitoring of claim 3, wherein said area comprises a first portion for decreasing said alarm volume level and a second portion for increasing said alarm volume level.

5. The system for patient monitoring of claim 1, wherein at least one light source is positioned on said rear face of said display.

6. The system for patient monitoring of claim 1, wherein said at least one patient monitor comprises a removable internal chassis for mounting a plurality of circuit boards.

7. The system for patient monitoring of claim 1, wherein said at least one patient monitor comprises a handle attached to said patient monitor and wherein said handle further comprises an up and a down position, a set-point for balancing said patient monitor perpendicular to the floor when said patient monitor is carried using said handle and, a damper to retard downward motion of said handle when said handle is released from the up position.

8. The system for patient monitoring of claim 1, wherein said at least one patient monitor comprises Li-Ion batteries and a microcontroller for monitoring charging, discharging and over-temperature conditions of said batteries.

9. The system for patient monitoring of claim 8, wherein said at least one patient monitor is capable of operating 8 hours on battery power while monitoring ECG, NIBP every 15 minutes and taking a recording every 15 minutes.

10. The system for patient monitoring of claim 9, wherein said at least one patient monitor weighs less than 9 pounds.

11. A docking station having a receiving surface to receive a monitor and display device of a patient monitoring system, said monitor and display device having a first connector and a first plurality of monitor receptacles for transmission of digital information and power, said docking station comprising:
a. a second plurality of receptacles;
b. a second connector positioned on said receiving surface of said docking station for mating with said first connector of said monitor and display device;
c. a circuit board for controlling said transmission of digital information and power;
d. a molded recess matching an external shape of a bottom portion of said monitor and display device;
e. at least one latching mechanism for securely holding said monitor and display device in place and,
f. a release button for disengaging said latching mechanism for removal of said monitor and display device from said docking station;
wherein, when said monitor and display device is securely mounted on said docking station via said latching mechanism, said first connector is in electrical communication with said second connector, further wherein said circuit board transfers said transmission of digital information and power away from said first plurality of receptacles, through said first and second connectors, and to said second plurality of receptacles.

12. The docking station of claim 11, wherein the said second plurality of receptacles comprises Ethernet connection, DVI for external display, USB, serial ports, external nurse alert/external audio/IR receiver, power and SDLC (synchronous data link control) port.

13. The docking station of claim 11, wherein said docking station covers said first plurality of receptacles on said monitor and display device when the said monitor and display device is docked in the docking station.

14. The docking station of claim 11, wherein said monitor and display device further comprises a first plurality of vents and said docking station further comprises a second plurality of vents, said first and second plurality of vents aligning when said monitor and display device is mounted in said docking station.

15. The docking station of claim 11, wherein said molded recess further comprises an outward bevel to guide the monitor and display device into position during docking.

16. The docking station of claim 11, further comprising at least one pin configured to fit at least one corresponding opening on said monitor and display device to further guide placement of said monitor and display device into said docking station.

17. The docking station of claim 11, wherein said release button is backlit when said monitor and display device is docked in said docking station.

18. An externally mountable pod for attaching to a monitor of a patient monitoring system, said pod comprising:
a. a plurality of pogo pins for mating with connectors on said monitor;
b. at least one guide pin for mating said pod with said monitor;
c. a latching mechanism for connecting and removing said pod to and from said monitor;
d. a button for actuating said latching mechanism; and,
e. a plurality of receptacles on a side of said pod.

19. The pod of claim 18, wherein said pod is a sidestream capnography or multigas pod.

20. The pod of claim 18, wherein said plurality of pogo pins enable the pod to receive power from said monitor and enable communication between said pod and said monitor.
